(19) United States
(12) Patent Application Publication
(10) Pub. No.: US 2008/0057089 A1
(43) Pub. Date: Mar. 6, 2008

(54) Light emitting personal lubricant technical field

(76) Inventor: Miguel Angel Molina, Napa, CA (US)

Correspondence Address: Law Offices of Gerald L. Robertson & Assoc., 1110 Castle Oaks Drive, Napa, CA, 94558-1576, US

(21) Appl. No.: 11/513,747

(22) Filed: Aug. 30, 2006

(51) Int. Cl. A61K 8/19

(52) U.S. Cl. 424/401

(57) ABSTRACT

Applicant's invention is a light emitting personal lubricant whose preferred embodiment comprises a combination of personal lubricant and luminescent particles such that when said particles are activated by a light source the personal lubricant takes on “glow in the dark” characteristics, or becomes light emitting.

### TECHNICAL FIELD

The present invention relates generally to personal lubricants, and more particularly to a speciality personal lubricant, and still more particularly to a personal lubricant composition providing luminescence, photoluminescence, bioluminescence, phosphorescence, or otherwise exhibiting a light emitting, “glow-in-the-dark,” quality. It does not function as a contraceptive, spermicide, or microbicide, though it may contain ingredients conductive to tissue healing, such as Vitamin E and/or aloe vera extract.

### STATEMENT OF GOVERNMENTAL ASSISTANCE

Applicant hereby represents that no part of the subject invention or application has come about with the assistance of government funds or by virtue of a government program.

### BACKGROUND INFORMATION AND DISCUSSION OF RELATED ART

The subject matter of the instant application was first disclosed in a Provisional Patent Application, Ser. No. 60/712,341, filed Aug. 30, 2005, and upon which priority date Applicant relies for this non-provisional application.

Personal lubricants are used to reduce genital or anal friction and irritation during medical procedures, such as ˜ecological examinations, digital rectal examinations, the insertion of enemas, suppositories, or rectal thermometers, or during sexual acts, such asintercourse or masturbation. Personal lubricants come in several types, including water soluble, oil based, silicone based, and organic.

Generally, sexual arousal is the primary stimulant for lubrication. In the case of female physiology, while female vaginal tissue does not contain secretory glands to provide lubrication in preparation for intercourse or other sexual play, it is highly vascularized, and the blood vessels in vaginal tissue become engorged with blood from sexual arousal. The blood vessels then put pressure on the vaginal tissue and induce it to excrete natural tissue fluids through the walls of the vagina. The excretions generally comprise small droplets of fluid disposed along the vaginal walls, primarily near the vaginal opening. With increased sexual excitement, however, this natural lubrication can entirely coat the sides of the vagina and largely provides the lubrication to facilitate intercourse, as male pre-seminal fluid scarcely makes a contribution. Without this natural lubricant, or an artificial one, a woman would most likely find penetration painful and possibly injurious.

On occasion, in some cases chronically, women fail to produce sufficient vaginal lubrication and the vaginal tissue becomes dry and irritated. Such dryness can be caused by a number of conditions, including a drop in estrogen levels from menopause or after hysterectomy, vaginal pathology, such as infection or cysts, or even radiation therapy. Drugs can also contribute to the problem. For instance, vaginal dryness can be caused by oral contraceptives high in progesterone, or by antihistamines, antidepressants, and medications for hypertension and cardiac conditions. Depression, stress, anxiety and other psychological factors are also causative by undermining arousal and thus the production of lubricating vaginal fluids. Finally, there are several conditions that produce general tissue drying or desiccation of membranous tissue. And these conditions can result in a dry mouth (xerostormia), dry eyes, dry nasal mucosa, dry rectalmucosa, dry skin, and, most pertinently, dry vaginal tissue.

Dryness is not alien to males, as well. In the context of homosexual relations, the use of personal lubricants is common as an aid in facilitating anal intercourse and mutual or reciprocal massage and stimulation. It is, of course, common for use when soloing sexually.

Because vaginal and mucossal tissue dryness is surprisingly widespread, and because a healthy sexual life is such a vital element in interpersonal relationships, a large industry has risen that is directed to the production and marketing of personal lubricants.

The type of personal lubricant currently preferred for use as an aid to sexual intimacy functions by providing water in either a gelled or viscous liquid form. The lubricant compositions generally include a water soluble cellulose derivative, such as methyl cellulose, hydroxyethyl cellulose, hydroxypropyl cellulose, sodium carboxymethyl cellulose. Alternatively, the lubricant may include water soluble polymers, including polyethylene oxide, polyethylene glycol, sodium alginate, polyvinyl pyrrolidone, polyvinyl alcohol, or a water soluble, silicon co-polymer, such as dimethecone, and similar substances.

The most common ingredients in the most popular brands of personal lubricants include: (1) Glycerin, which is chemically similar to sugar and has a sweet taste. (2) Polymers (such as those identified above), which are biologically inactive plastics that primarily function as emulsifiers or thickeners and provide a viscous, thick and slick feel. They are hydrophilic and retain substantial volume, which can then be released with rubbing. (3) Next in order of appearance is Nonoxynol-9, a spermidical detergent. (4) Preservatives, the most frequently found being parabens. Grapefruit seed extract and sodium benzoate are also used as preservatives. (5) Aloe vera extract, well known for its healing properties. And (6) deionized (“DI” ) or purified water. Virtually all water based personal lubricants are based on DI water.

At present there are scores of personal lubricants on the market, the naming and branding for which has become quite explicit and clearly intended to evoke sexual images in the mind of the consumer. Popular non-water based versions include: Vibrel for Women; Yes! For Women Cream; Adam & Eve; Probe; Slippery Stuff; Erogel; Astroglide; and Wet. A few of themore popular water based lubricants include: Astroglide; Astrogel; Wet; Trojan Lube; K-Y Jelly;Slippery When Wet; Hydra Smooth; System JO H20Lube; Body Action Ultra Light; Body Action Prolong; Body Action Supreme; Forplay; EROS; Sex Grease; Liquid Sex; Smooth &Slick; ID Glide; Sensual Moist; Elbow Grease Encounter; Ultra Lube; Climax Lube; Sylk; SensuaOrganics; Boy Butter; Empowered Products Stroke 29 Rocket Fuel; Sliquid Silk; Motion Lotion; Wet-n-Ready; Nudelube; AnalEase; AnalLube; Boi Toyz Anal Lube; Rear Entry; Jack Jelly Head Spread; Hand Job; Pjur Eros Premium Power; and Bliss Lube—to name just a few. The water-based personal lubricants are preferred because they are generally the least irritating to skin and mucossal tissue.

Personal lubricants can also be pleasantly flavored. Some even exploit transdermal technology and are specifically tailored to produce desired physiological effects and sensations, in addition to adding lubricity. For instance, some personal lubricants cause a increased tactile sensitivity, a warming sensation, or vasodilation upon skin contact. These can be used to enhance penile or clitoral erection.

The foregoing prior art lubricants reflect the current state of the art of which the present inventor is aware. Reference to, and discussion of, these products is intended to aid in discharging Applicant's acknowledged duty of candor in disclosing information that may be relevant to the examination of prospective claims to the present invention. However, it is respectfully submitted that none of the above-indicated products disclose, teach, suggest, show, or otherwise render obvious, either singly or when considered in combination, the invention described herein. Specifically, none of the foregoing personal lubricants emit or reflect light, either as a result of being energized by light, heat, electricity, or by being combined with other substances.

Accordingly, it is an object of the present invention to provide a novel light emitting personal lubricant that may be either phosphorescent, luminescent, photoluminescent, bioluminscent, chemiluminescent, reflective or otherwise “glow-in-the-dark.”

It is another object of the present invention to provide a novel personal lubricant for the lubrication of vaginal or rectal tissue to facilitate comfortable penetration during intercourse.

It is still another object of the present invention to provide a novel personal lubricant that provides visually erotic stimulation.

Yet another object of the present invention is to provide a personal lubricant that emits light.

Still another object of the present invention is to provide a personal lubricant which reflects light.

A still further object of the present invention is to provide a light emitting personal lubricant that is not radioactive.

Another object of the present invention is to provide a personal lubricant that possesses tissue healing properties.

A further object is to provide a water soluble, non-staining personal lubricant with increased viscosity and lubricity.

Other novel features which are characteristic of the invention, together with further objects and advantages thereof, will be better understood from the following specification, in which preferred embodiments of the invention are described by way of example. It is to be expressly understood, however, that the examples are for description only and are not intended as a definition of the limits of the invention. The various features of novelty that characterize the invention will be identified with particularity in the claims appended hereto. The invention does not reside in anyone of these features taken alone, but rather in the particular combination of all of its elements.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other compositions, methods, and systems for carrying out the several purposes of the present invention. It is important, therefore, that the prospective claims be regarded as including such equivalent compositions insofar as they do not depart from the spirit and scope of the present invention.

### DETAILED DESCRIPTION OF THE INVENTION

The personal lubricant of the present invention is a light emitting personal lubricant. The light emitting quality can be of virtually any type, including phosphorescence, luminescence, photoluminescence, bioluminscence, or chemiluminescence. In a preferred embodiment of the inventive composition, however, the lubricant is photoluminescent, and thus exhibits light emitting characteristics after itself first having been energized by exposure to light. To achieve the desired illumination, any of a number of suitable photoluminescent particles or pigments can simply be added to the well known compositions referred to above by brand name. It will be appreciated, however, that certain phosphors are known to work well as “glow-in-the-dark” substances, and these are preferred additives that give the present invention its novel advantages. The use of the term “pigment” herein is intended to suggest an additive which will allow a color to form in the lubricating medium. The use of this term should not be mistaken for the common meaning of pigment as used to denote a coloring agent for paints and dyes except as it relates to one embodiment of the invention.

In the traditional sense, a pigment may be employed in a personal lubricant such that the pigment would become luminous as a result of its reflection of or appearance under certain lighting conditions. For example, a white pigment of suitable composition which would illuminate when subjected to ultraviolet or “black” light thereby making any part of the body coated in the lubricant glow a bright color could be used as one embodiment of the instant invention. This embodiment though does not have the convenience of the preferred embodiment in that the lubricant will only glow under exposure to ultraviolet light. The preferred embodiment instead, having been charged by a light or energy source will glow without the need for further light or spectra.

It is well known to employ alkaline earth metal aluminate in a powder form to provide glow-in-the-dark effect for transparent and semi-transparent products, such as plastic toys, or glass exit lighting. The alkaline earth aluminates are commonly referred to as or commonly called strontium aluminate, silicate aluminate, or alkaline earth aluminate. A number of earth metals can be used, depending on the particular product and color desired., including strontium, magnesium, calcium, and barium, to make, for example, barium aluminate, calcium aluminate, and magnesium aluminate. The glowing colors span the spectrum from greenish yellow to purple blue. Silicon or titanium may be added, and each alkaline earth metal aluminate may be doped with europium. Further, alkaline earth silicates can be employed, as can silicate aluminate and zinc sulfide, though with notably less luminosity and persistence than strontium aluminate. Alkaline earth silicate produces a sky blue color not produced by alkaline earth aluminates. Yet another alternative is earth mineral crystals.

The alkaline earth aluminate materials can be coated with a sealer to render them insoluble in water, which makes them suitable for use in water-based personal lubricant compositions.

When provided as a photoluminescent composition, the personal lubricant of the present invention requires some exposure to light before use. However, the light emitting quality of the lubricant thereafter persists for hours at sufficient intensity to fully illuminate and present sexual play. The partners can freely and selectively spread the lubricant all over themselves and each other as desired. Then, in an otherwise dark room, shadows and silhouettes become prominent erotic features. A penis or a breast may stand out like a lighthouse, yet the soft light emitted will only outline a lover's form.

In a preferred composition, the personal lubricant of the present invention comprises about 30% glycerin, about 5% propylene glycol, about 10% glyceryl polymethacrylate, about 5% pentylene glycol, about 5% PPG-51/SMDI copolymer, about 8% photoluminescent pigment, about 5% water soluble cellulose gum, about 5% preservative, and about 35% water. This composition has a lubricity in the range of about 33-466.

In a second preferred embodiment of the personal lubricant of the present invention, the composition comprises 5-50% by weight glycerin, 0-40% by weight of propylene glycol, 5-25% of sorbitol, 0.25-1.0% by weight of water soluble cellulose gum. This composition has a pH in the range of 4-5 and a lubricity in the range of about 33-466.

In other embodiments, the luminescent material, whether pigment or particles, may be added to brand name personal lubricants already offered for sale. In effect, the brand name product simply functions as the carrier for the luminescent pigment. Ingredients in an exemplary water based lubricants might include: water, glycerin, ethoxydiglycol, guarhydroxypropyltrimonium, chloride, hydroxyethylcellulose, citric acid, benzyl alcohol, methylisothiazolinone, and methylchloroisothiazolinone, in any combination.

The ingredients in an exemplary non-water based lubricant might include deionized water, mineral oil, petrolatum, paraffin, sorbitan stearate, plysorbate-60, stearic acid, stearyl alcohol, ceteareth-20, cetyl alcohol, lanolin, borax, bethylparaben, propylbaraben, and imidazolidiny Urea. Alternatively, a simple composition carrier could include dimethicone, dimethiconol, and cyclomethicone. Other suitable ingredients include cellulose thickener, and propylene glycol.

The preferred particle size for luminescent particles is between 5 and 120 μm.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, arid operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

I claim:

1. A light emitting personal lubricant comprising:
personal lubricant such as the kinds and types used for medical procedures and examinations including insertion of objects into the body, massage and sexual acts, said personal lubricants generally being water soluble, oil based, silicone based, or organic;
light emitting pigment, such that when said pigment is introduced into said personal lubricant, said personal lubricant becomes light emitting.

2. The invention described in claim 1 wherein:
said light emitting pigment is comprised of phosphors.

3. The invention described in claim 1 wherein:
said light emitting pigment comprising powdered alkaline earth metal aluminates, said aluminates selected from the group of strontium aluminate, silicate aluminate and alkaline earth aluminate.

4. The invention described in claim 3 wherein said alkaline earth metal aluminate is doped with europium.

5. The invention described in claim 3 wherein said light emitting pigment is coated with a sealer to render said pigment insoluble in water.

6. The invention described in claim 1 wherein:
said light emitting pigment selected from the group of alkaline earth silicate, silicate aluminate, zinc sulfide and earth mineral crystals.

7. The invention described in claim 1 wherein:
said personal lubricant comprising about 30% glycerin, about 5% propylene glycol, about 10% glyceryl polymethacrylate, about 5% pentylene glycol, about 5% PPG-51/SMDI copolymer, about 8% photoluminescent pigment, about 5% water soluble cellulose gum, about 5% preservative, and about 35% water, said personal lubricant having a lubricity in the range of about 33-466.

8. The invention described in claim 1 wherein:
said personal lubricant comprising 5-50% by weight glycerin, 0-40% by weight of propylene glycol, 5-25% of sorbitol, 0.25-1.0% by weight of water soluble cellulose gum, said personal lubricant having a pH in the range of 4-5 and a lubricity in the range of about 33-466.

9. The invention described in claim 1 wherein:
said personal lubricant comprising deionized water, mineral oil, petrolatum, paraffin, sorbitan stearate, plysorbate-60, stearic acid, stearyl alcohol, ceteareth-20, cetyl alcohol, lanolin, borax, bethylparaben, propylbaraben, and imidazolidiny Urea.

10. The invention described in claim 1 wherein:
said personal lubricant comprising dimethicone, dimethiconol, and cyclomethicone.

11. The invention described in claim 10 further comprising:
cellulose thickener and propylene glycol.

12. The invention described in claim 1 wherein:
said personal lubricant comprising water, glycerin, ethoxydiglycol, guarhydroxypropyltrimonium, chloride, hydroxyethylcellulose, citric acid, benzyl alcohol, methylisothiazolinone, and methylchloroisothiazolinone, in any combination.

13. The invention described in claim 1 wherein:
said pigment comprising luminescent particles, the particle size for said luminescent particles being between 5 and 120 μm.

14. The invention described in claim 1 wherein:
said personal lubricant becomes light emitting after being sufficiently exposed to light.

15. A light emitting personal lubricant comprising:
personal lubricant such as the kinds and types used for medical procedures and examinations including insertion of objects into the body, massage and sexual acts, said personal lubricants generally being water soluble, oil based, silicone based, or organic;
pigment for combination with said lubricant such that when said lubricant and pigment combination is subjected to ultraviolet or “black light”, said combination illuminates.
